(19) United States
(12) Patent Application Publication
(10) Pub. No.: US 2015/0305727 A1
(43) Pub. Date: Oct. 29, 2015

(54) KITS AND METHODS FOR VISUALIZING A CARDIAC CHAMBER FOR CONNECTION TO A MECHANICAL CIRCULATORY SUPPORT DEVICE

(71) Applicant: THE CLEVELAND CLINIC FOUNDATION, Cleveland, OH (US)

(72) Inventors: Jamshid Karimov, Cleveland Hts, OH (US); Kiyotaka Fukamachi, Mayfield Hts, OH (US); Nader Moazami, Moreland Hills, OH (US)

(21) Appl. No.: 14/691,969

(22) Filed: Apr. 21, 2015

Related U.S. Application Data
(60) Provisional application No. 61/984,589, filed on Apr. 25, 2014.

(51) Int. Cl. A61B 17/00; A61M 1/12

(52) U.S. Cl. CPC A61B 17/0057; A61M 1/122; A61B 2017/00672; A61B 2017/00654

(57) ABSTRACT

Systems, kits, and methods that provide direct visualization of a patient's surgical field and intracardiac structures during implantation of a mechanical circulatory support device. Kits and methods can provide for seamless implantation of such devices, which reduces the risks and time associated with mechanical circulatory support device implantation. Further, methods are provided that can avoid cardiopulmonary bypass.

### RELATED APPLICATIONS

This application claims the benefit of U.S. Provisional Patent Application Ser. No. 61/984,589, filed Apr. 25, 2014, the entirety of each of which is hereby incorporated by reference for all purposes.

### TECHNICAL FIELD

The present invention generally relates to kits and methods for visualizing a bloodless surgical field during or after connection of a mechanical circulatory support device.

### BACKGROUND

More than sixteen million people are currently diagnosed with chronic heart failure (CHF) in the United States and Europe, where its prevalence averages 2.5% of the normal population. CHF increases significantly after age 65 and the population in this group will double within the next 20 years, suggesting heart failure incidence will similarly increase. Heart transplantation is still the therapy of choice for patients with sustained heart failure resistant to any medical therapy. Need for heart transplantation or mechanical circulatory support is estimated to be 50,000 to 100,000 patients per year. However, only 2,000 to 3,000 cases of heart transplantation are performed per year. In the last decades, long waiting times for cardiac transplantation and subsequent increased mortality have led to an increase in the use of mechanical circulatory support devices, such as left ventricular assist devices (LVADs). There is a 25% annual growth of the number of LVAD implants worldwide (1,990 cases in 2007 and 4,650 cases in 2011) and it is estimated that there may be approximately 10,000 LVAD implants in 2015.

During the last decades, vast improvements in mechanical circulatory support devices resulted in a breakthrough of ventricular assist device (VAD) technology, whose attributes are transforming this therapy into a standard of care for end-stage heart failure. Permanent mechanical circulatory support by smaller devices is a promising therapeutic option developed to provide an alternative to transplantation and to reduce mortality on the heart waiting list. VADs have already been proven to provide excellent circulatory support and to improve survival until heart transplantation. Despite these advances, many patients still suffer significant morbidity or even death in association with the implantation procedure itself. Patients are still referred for this therapy fairly late in the course of therapy for heart failure as some still view this therapy as an option of last resort. Implantation techniques that decrease operative morbidity and mortality are important for the continued advancement of the field. One of those aspects is the device implantation technique, which is primarily based on putting a patient on cardiopulmonary bypass (CPB) (extracorporeal pump) and implantation of the dedicated pump (VAD) inside the patient's chest.

The visualization of intra-cardiac structures before the VAD implant is an important step in surgical implantation of the device. The visualization of the heart structures inside the left ventricle (LV), for example, is possible but requires the institution of the CPB to open the heart. This is an important limitation in these procedures, which significantly may worsen clinical outcomes due to the deleterious effects (complications may exacerbate multiple organ failure and increase blood transfusions during and after the operation) of the CPB itself. Also, the total operating times are increased with the use of CPB, in addition to elevated costs for the materials, equipment and personnel involved in the CPB operation and potential hazards of cannulation. Also, after the procedure, the CPB cannulae and lines should be withdrawn and insertion sites checked for bleeding. The cannulation sites are considered potential bleeding points in the early post-operative period that may require a re-operation. Any extra manipulation during a major surgery requires extra attention and efforts for the surgical team and is associated with prolonged total operative time and increased material costs.

There is currently no method for off-pump (without CPB) VAD implantation that allows a surgeon to visualize the intraventricular structures in a bloodless surgical field. Further, there is no method or system where the VAD can be connected to the LV, for example, without any interruption of the circulatory circuit. There are current methods where surgeons are able to manage an off-pump implementation of a VAD using handcrafted techniques (such as a surgeon using his finger as an obturator of the LV hole, using a Foley catheter as an occluder, quicker connection of the VAD after making the hole in the LV, or using an endoventricular occlusion balloon, or a dedicated plug that may come with the VAD). Although the methods could be off-pump, they do not allow a complete and controlled visualization of the surgical field because the cardiac ventricles are filled with blood. Also, there is currently no method to visualize the internal chamber of the ventricle when a VAD has been removed, and/or before the VAD has to be replaced with another device. Presence of clots in the ventricle or muscle bands that will obstruct the VAD may be deleterious if the device is inserted blindly. In cases of device removal (as may be necessary if the patient's heart has recovered or for other reasons such as infection or a clot in the device), visualization of the inside of the cardiac chamber is important to ensure that no residual clots or other material is left behind in the heart after the device has been removed.

### SUMMARY

Embodiments of the present invention generally relate to visualizing the surgical field during connection of a mechanical circulatory support device in a patient. In an embodiment, a cardiac chamber visualization kit is provided. A kit comprises an occlusion system comprising a catheter having a distal portion and a proximal portion and at least one lumen extending therein. The system further comprises a distal occluder mounted on the distal portion of the catheter. The system also includes a proximal occluder mounted on the proximal portion of the catheter. The kit further comprises a mechanical circulatory support device.

In another embodiment, the present invention provides a method for visualizing a cardiac chamber for insertion or removal of a mechanical circulatory support device in a patient. The method can be used to visualize a cardiac chamber before, during, and/or after insertion of a mechanical circulatory support device. For example, the method can be used for visualizing an interior cardiac chamber after removal of the mechanical circulatory support device. A method comprises providing an occlusion system as described above. The method further includes introducing a cannula into a cardiac chamber (such as an atrial appendage, ventricle, atrium, or any suitable combination thereof) of the patient via a cardiac wall of the patient (such as, for example, a ventricular wall or ventricular apex at any suitable location). The occlusion system is then inserted into the cannula with the distal and proximal occluders in a contracted state. The method further includes introducing the distal and proximal occluders into the cardiac chamber and expanding the distal and proximal occluders sequentially or simultaneously into an operative configuration. The cardiac wall (such as the ventricular apex or a ventricular free wall, or any other suitable cardiac chamber) is then cored and the cardiac chamber is de-aired. The distal and proximal occluders are removed from the cardiac chamber and the mechanical circulatory support device is connected to the cored cardiac wall via an inflow cannula of the mechanical circulatory support device.

### BRIEF DESCRIPTION OF THE DRAWINGS

FIG. 1 is a perspective view of an occlusion system according to an embodiment of the present invention with the occluders in a partially deflated state.

FIG. 2 is the occlusion system of FIG. 1 with the occluders in an inflated state.

FIG. 3 is a cross-sectional view of the occlusion system of FIG. 2 along lines 3-3.

FIG. 4 is a cross-sectional view of another embodiment of an occlusion system of the present invention.

FIG. 5 is a side view of an embodiment of an occlusion system of the present invention connected to an infusion source with the occluders partially expanded in the context of a cross-sectional partial schematic illustration of the heart.

FIG. 6 is a side view of the occlusion system of FIG. 5 with the occluders in an operative configuration in the context of a cross-sectional partial schematic illustration of the heart.

FIG. 7 is a side view of the occlusion system of FIG. 6 with a sheath positioned in the ventricular apex of the heart in the context of a cross-sectional partial schematic illustration of the heart.

FIG. 8 is a side view of the sheath depicted in FIG. 7 connected to a VAD via a VAD inflow cannula in the context of a cross-sectional partial schematic illustration of the heart.

FIG. 9 is a flow chart indicating steps of a method according to an embodiment of the present invention.

### DETAILED DESCRIPTION

The present invention provides kits, systems and methods for visualizing a cardiac chamber of a patient during insertion of a mechanical circulatory support device. Although, the present invention will be described with respect to visualizing a ventricle, any cardiac chamber can be visualized with the kits, systems and methods of the present invention such as an atrium or atrial appendage. Further, although the disclosure below refers to a VAD as an exemplary mechanical circulatory support device, any suitable mechanical circulatory support device can be used. A mechanical circulatory support device includes a biomechanical circulatory support device. Further, although the disclosure is described mainly with respect to insertion/removal of a left VAD (LVAD) into a left ventricle, the kits, systems and methods can be used for insertion of a right VAD (RVAD) into a right ventricle, a bilateral VAD (BiVAD) into both ventricles or insertion of another mechanical support device into a cardiac chamber of the patient. The disclosure herein also refers to the term “substantially” with respect to certain geometric shapes. By “substantially” is meant that the shape of the element need not have the mathematically exact described shape but can have a shape that is recognizable by one skilled in the art as generally or approximately having the described shape. Also, the disclosure herein refers to an “operative configuration.” An operative configuration refers to the configuration of the occlusion system when the occluders are fully deployed. The occluders are fully deployed when they are in the necessary cardiac chamber of the patient and they are each contacting the inner surface of the cardiac chamber to create a mechanical barrier to blood flowing to the insertion site of the device from the cardiac chamber. The disclosure also refers to the terms “right” and “left.” These terms refer to anatomical directions and orientations of a human patient in a standard anatomical position as is known in the art.

Further, as used herein with respect to a described element, the terms “a,” “an,” and “the” include at least one or more of the described element unless otherwise indicated. Further, the term “or” refers to “and/or” unless otherwise indicated. In addition, it will be understood that when an element is referred to as being “on,” “attached” to, “connected” to, “coupled” with, “contacting,” in “communication” with etc., another element, it can be directly on, attached to, connected to, coupled with, contacting, or in communication with the other element or intervening elements may also be present. In contrast, when an element is referred to as being, for example, “directly on,” “directly attached” to, “directly connected” to, “directly coupled” with, “directly contacting,” or in “direct communication” with another element, there are no intervening elements present. It will also be appreciated by those of skill in the art that references to an element that is disposed “adjacent” another element may have portions that overlap or underlie the adjacent element.

Aspects of the present invention allow a practitioner to visualize intracardiac structures, such as intraventricular structures, for controlled insertion of a VAD. Methods and kits as described herein can be used without CPB (i.e. an off-pump procedure and/or on a beating heart with some level of circulatory support) and provide a direct surgical visualization by maintaining a bloodless surgical field and preferably a seamless (uninterrupted) device connection to the heart. The off-pump application reduces complications associated with the use of CPB. The visualization in a bloodless field provides a better view of the working field, better control of the inflow cannulation at the ventricular apex or other cardiac wall into which the inflow cannula is inserted, and minimizes mismatches of devices related to poor visibility and access. In preferred embodiments where there is a seamless device connection, such a connection provides improved device and ventricular chamber de-airing, lessens the potential for air embolism, and seals the VAD insertion site. The methods described herein can be used for the first time when a VAD is connected to the heart, or during a VAD replacement for different indications. Direct visual control of the insertion site may also be helpful in preventing various complications associated with device insertion into the cardiac chamber.

The use of instrumental methods such as ultrasound (transesophageal, transthoracic echocardiography, Doppler methods) and imaging techniques (fluoroscopy, x-ray and other methods) may provide additional information during the deployment of the occluders to ensure exact positioning of the device inside the cardiac chamber. In addition, by using instrumental methods, it can be ensured that the flow has been successfully interrupted by the occluders and the cardiac chamber is isolated from the “working field.”

As with any cardiac surgical operation, the method in which the surgical device is inserted can have an impact on short-term and long-term outcomes associated with the procedure. Attention to details in the surgical technique is important and can affect post-operative risks, proper device function, and standard complications associated with prolonged operative times. Since certain methods of the present invention can be used without CPB, negative effects on a patient's physiology and hospital costs associated with the use of CPB can be eliminated. Also, shorter operative times are expected with use of embodiments of the present invention compared to current surgical VAD implantation techniques.

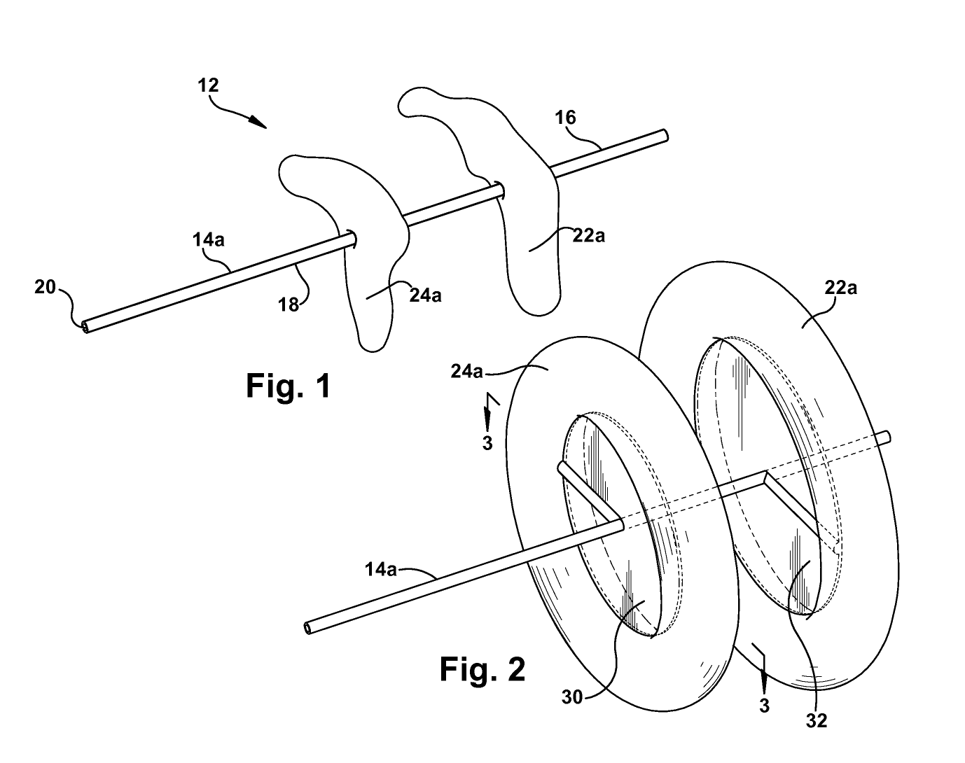

Referring to FIG. 1, an embodiment of the present invention provides a cardiac chamber visualization kit comprising an occlusion system 12. Occlusion system 12 comprises a catheter 14 having a distal portion 16 and a proximal portion 18 and at least one lumen 20 extending therein. System 12 further includes a distal occluder 22 disposed on distal portion 16 of catheter 14 and a proximal occluder 24 disposed on proximal portion 18 of catheter 14. FIG. 1 illustrates only two occluders but the kit can include more than two occluders.

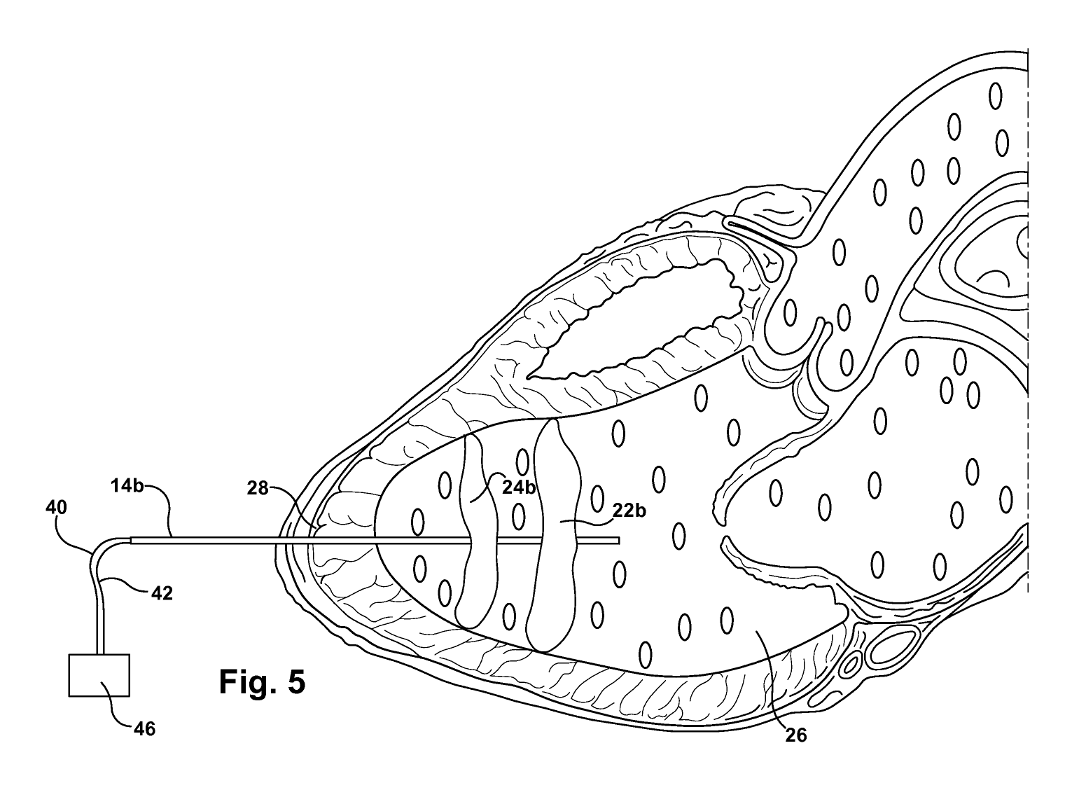

With reference to FIG. 5, an occluder can be any suitable device that contacts the inner surface of the LV 26 or other relevant cardiac chamber and provides a mechanical barrier to blood coming to the LV apex area 28. This barrier formation prevents blood flow towards the surgical working field (e.g. towards the apex where the LVAD is connected to the LV) and thus enables direct visualization inside the LV. An occluder can be any suitable device that is capable of expanding or de-compressing and contracting or compressing. For example, the occluder can be an inflatable balloon, a bladder, or an umbrella or parachute-like device. In the case of an occluder that has a hollowed interior that accepts a material to inflate the occluder (e.g. an inflatable occluder), the inflatable occluder can be inflated or diluted by delivering a fluid or air to the interior of the occluder. FIG. 1 illustrates partially inflated occluders 22 and 24 and FIG. 2 illustrates fully inflated occluders 22 and 24. Similarly, the inflatable occluder deflates upon removal or suction of the inflating material. An inflatable occluder can have any suitable configuration that provides a mechanical barrier to blood coming to the cardiac wall to which an inflow cannula or other conduit to a mechanical circulatory support device is connected, such as, for example, the LV apex area. For example, the inflatable occluder can be cylindrical, cone-shaped, or doughnut-shaped in an operative configuration (e.g. fully inflated). The occluder can have any suitable shape such as, for example, a barbell shape, an hourglass shape, a spheroidal shape, an ellipsoidal shape, an egg-shape, or any other non-uniform or asymmetric shape or asymmetric expandable unit so long as the occluder occludes blood flow to the working field. In the case of a doughnut shaped configuration, the inflatable occluders are effectively rings surrounding a mesh or fabric material 30 and 32 as illustrated in FIG. 2. In such an embodiment, less inflating material is delivered to the expandable portion of the inflatable occluder and the catheter size is smaller. In the case of the occluders being inflatable occluders such as balloons, for example, the inflatable occluders are expanded by introducing a fluid or other infusion material into the inflatable occluders to cause the inflatable occluders to expand. The occluders can have a roughened outer textured surface for better grip and to prevent dislodgement/sliding within the LV.

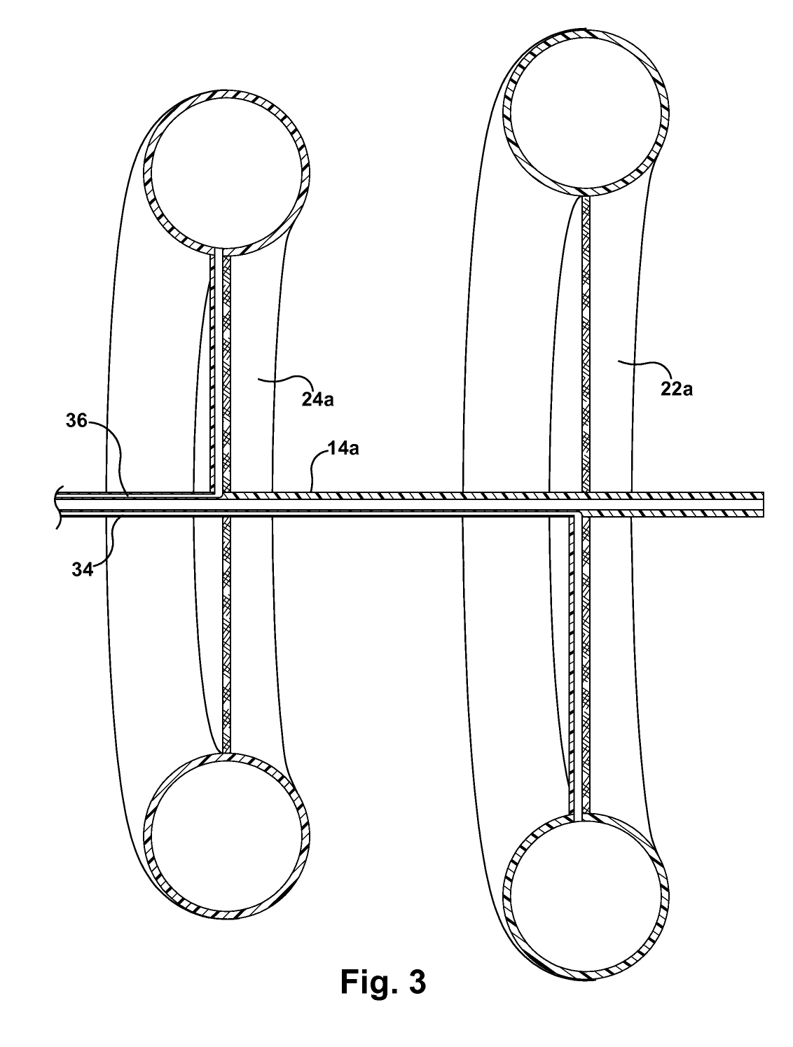

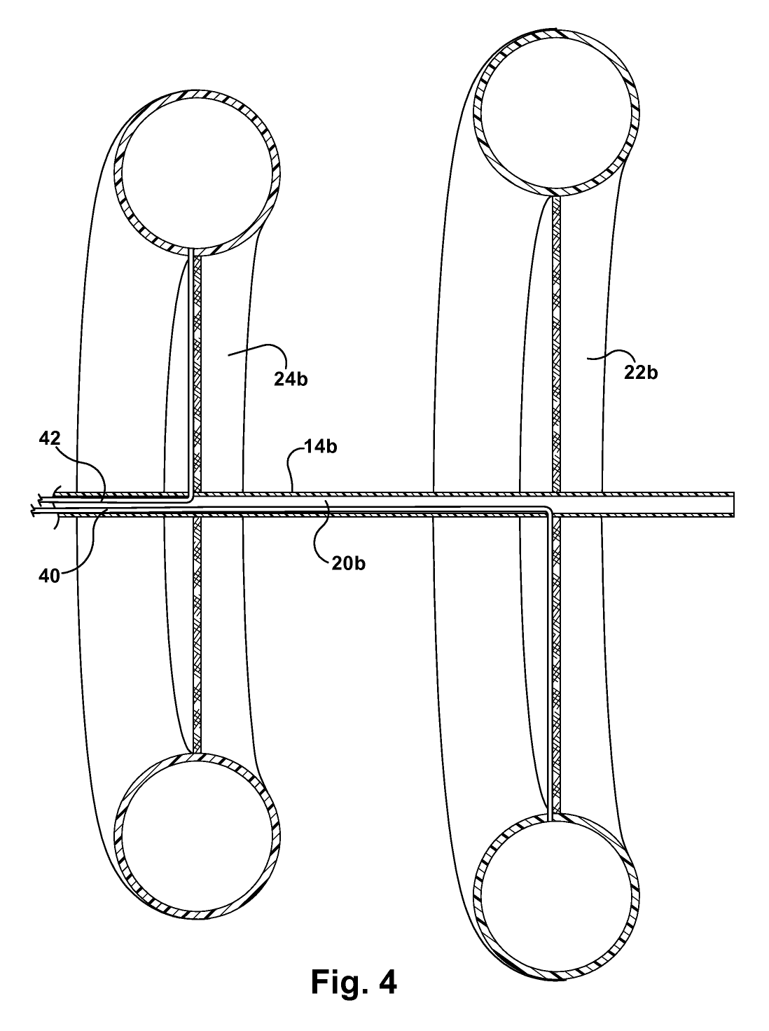

The occluders can be inflated in a variety of ways. Referring to FIG. 3, in certain embodiments, catheter 14a has at least two lumens. For example, first lumen 34 is in fluid communication with an infusion source (not shown) at one end and distal occluder 22a at another end. Catheter 14a further includes second lumen 36 in fluid communication with the infusion source at one end and proximal occluder 24a at another end. Alternatively, with reference to FIG. 4, lumen 20b of catheter 14b can include first infusion line 40 extending in lumen 20b. First infusion line 40 is in fluid communication with an infusion source 46 (depicted schematically in FIG. 5) at one end and distal occluder 22b at another end. Lumen 20b of catheter 14b further includes second infusion line 42 in fluid communication with the infusion source at one end and proximal occluder 24b at another end.

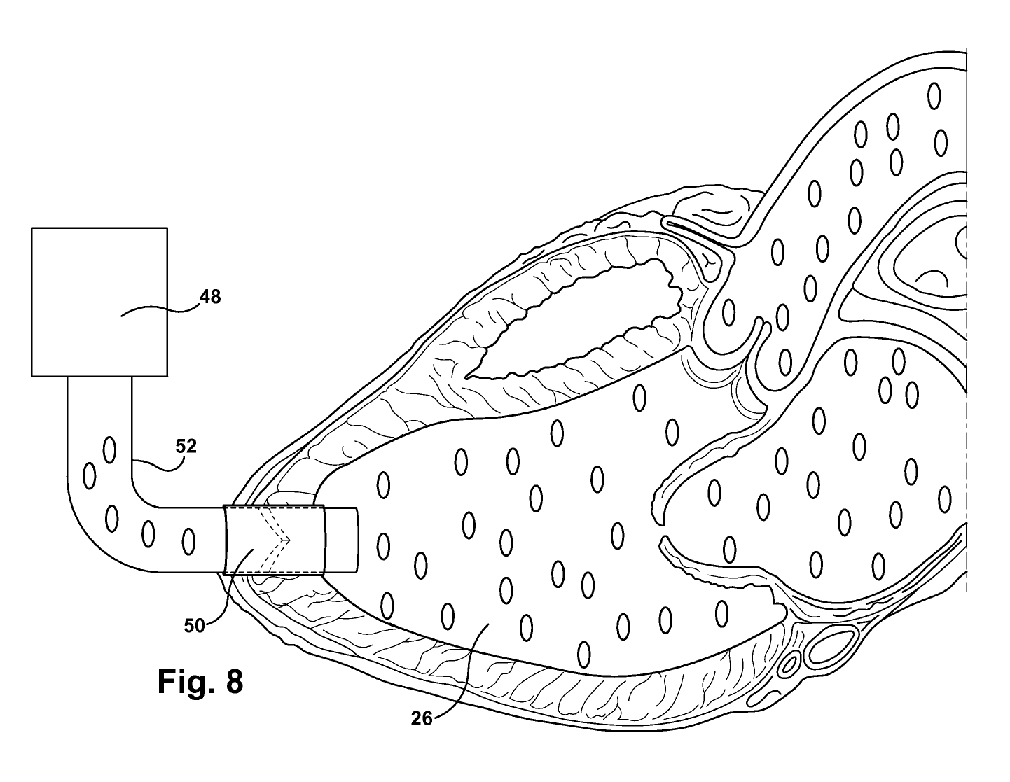

The cardiac chamber visualization kit also includes any VAD of suitable configuration 48 as schematically illustrated in FIG. 8. The VAD can be an LVAD, an RVAD, BiVAD, or another device which supports the circulation mechanically or biomechanically.

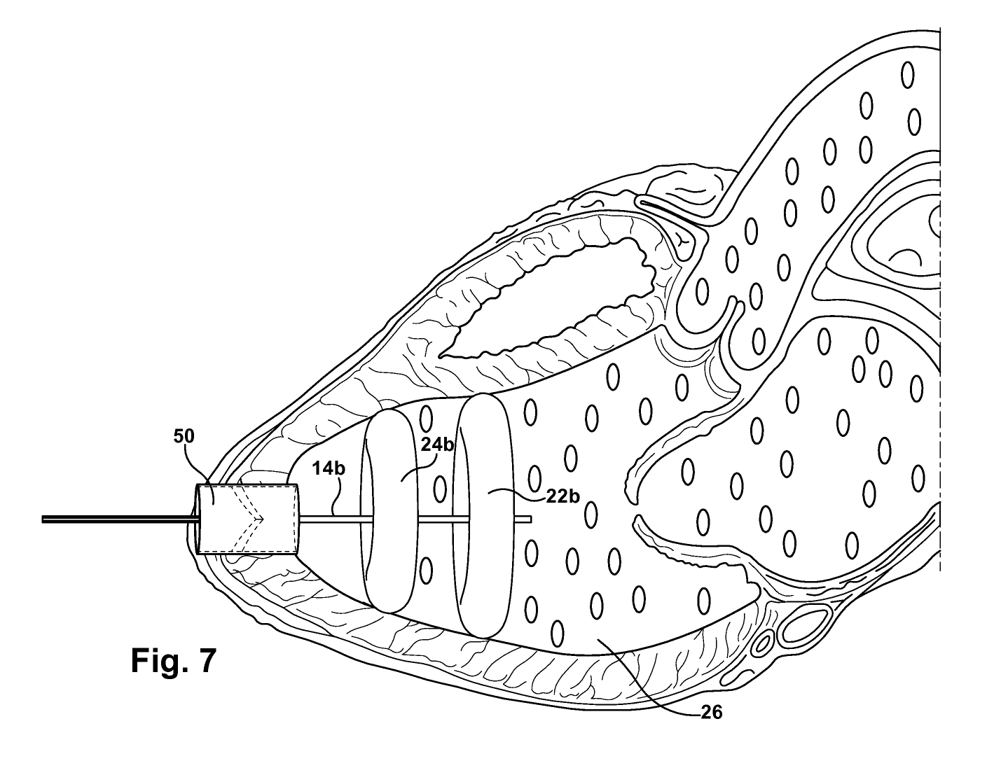

Referring to FIGS. 7 and 8, in certain embodiments, a kit of the present invention includes a sheath 50 that is inserted in the ventricular apex or other cardiac wall to which a conduit to a mechanical circulatory support device is attached. The sheath is a hemostatic valve to seal the ventricle from air entrapment and fluid leakage. Thus, the sheath can provide a seamless (uninterrupted) connection to the VAD inflow cannula 52 (schematically illustrated in FIG. 8) to improve cardiac de-airing and thus lessen the potential for an air embolism and minimize blood loss.

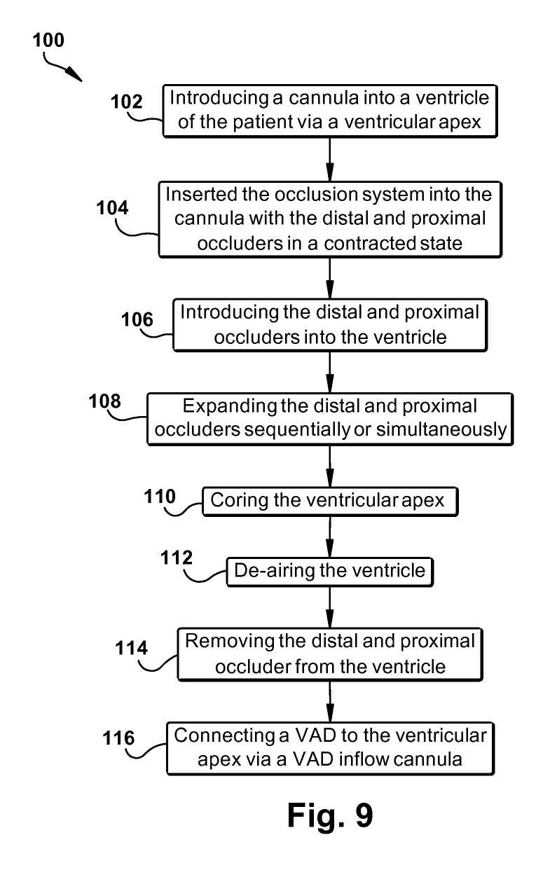

In an embodiment, the present invention provides a method of providing intraventricular or intracardiac visualization during insertion of a VAD or other mechanical circulatory support device. With reference to FIGS. 5-8 and flow chart 100 of FIG. 9, an exemplary method 100 comprises introducing a cannula into a cardiac chamber of a patient in need thereof, such as ventricle 26 via a cardiac wall, such as ventricular apex 28 that will be in communication with the inflow cannula 52 of the VAD 48 or other mechanical circulatory support device (such as, for example, the left ventricular apex) (102). Then the occlusion system including catheter 14b and occluders 22b and 24b is inserted into the cannula with the occluders 22b and 24b in a contracted or compressed state (104). The occluders are then introduced into a ventricle or other cardiac chamber 26 as schematically illustrated in FIG. 5 (106). Once occluders 22b and 24b are properly positioned in the cardiac chamber such as ventricle 26, the occluders are expanded or de-compressed (108). The distal occluder can be deployed first and then the proximal occluder can be deployed. Alternatively, the occluders can be deployed simultaneously. For example, in the case of inflatable occluders such as balloons, the distal occluder first can be inflated and then the proximal occluder can be inflated. Alternatively, the occluders can be simultaneously inflated. The occluders can be repositioned if necessary during the surgical procedure.

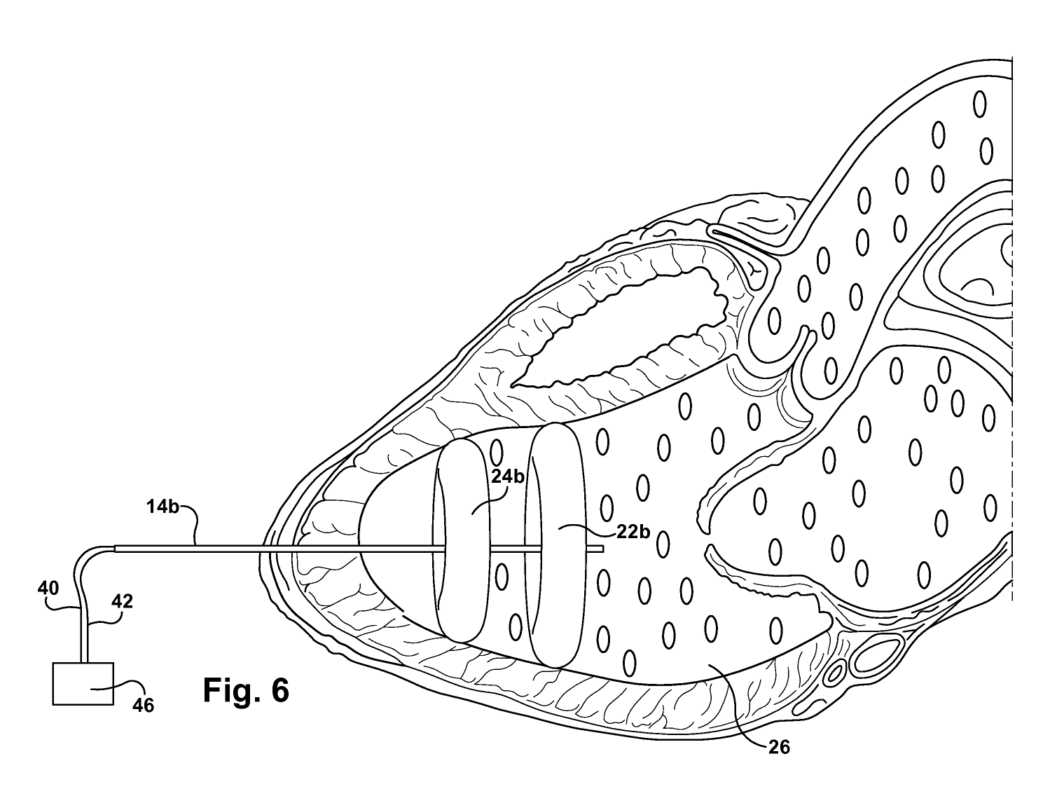

Before a ventriculotomy is performed and the occluders assume an operative configuration, sutures for a VAD inflow sewing ring can be placed around the ventriculotomy site. The VAD fixation may be performed by means of a sutured sewing ring to which the VAD (or its inflow cannula) is connected (e.g. inserted, snapped, locked, fixed, snared by and/or tightened). This can minimize the time the occluders need to be in place and can facilitate faster implantation of the VAD. Subsequently, the occluders are deployed in an operative configuration (e.g. fully expanded or fully de-compressed) as schematically illustrated in FIG. 6. This prevents blood (depicted by ovals in the figures) from flowing towards the surgical working field towards the apex or other cardiac wall providing direct visualization inside the ventricle. The physiologic blood flow within the heart chambers may be temporarily altered by the device expanded within the chamber. However, the natural blood flow and its direction are preserved for the whole duration of the VAD implantation.

A proximal and distal occluder is used to stabilize the occlusion system and to avoid dislodgement inside the ventricle. In other words, the position of one occluder is supported by another and vice versa. Two or more occluders are also preferable to provide a better barrier to blood flow. Further, two or more occluders provide a safeguard in case one occluder fails to close the blood flow and/or one occluder ruptures during the procedure (e.g. there will still be another occluder remaining in place).

When a totally bloodless field is obtained, the ventricular apex is cored with a standard cutter (110). The necessary trabeculae are removed through the ventriculotomy under direct vision. In embodiments including a sheath as described above, sheath 50 is introduced through the incision in the apex as schematically illustrated in FIG. 7. Such a sheath is a hemostatic valve that seals the ventricle from air entrapment and fluid (e.g. blood leakage).

The ventricular chamber is de-aired (the air entrapped in the ventricular chamber is removed) (112). The ventricle may be filled through the sheath with fluids and/or the occluders may be gradually contracted or compressed (e.g. deflated in the case of balloons) thereby restoring natural blood flow to the ventricle. The occluders then can be removed from the ventricles through the sheath, for example (114).

Referring to FIG. 8 and FIG. 9, after the occluders are removed from the cardiac chamber, such as ventricle 26 and the heart is successfully de-aired, VAD 48 or another mechanical circulatory support device via the VAD inflow cannula 52 or other conduit is connected to the apex or other cardiac wall (116). In the case of embodiments including a sheath, the VAD inflow cannula can be connected to the sheath. Since the ventricle was effectively de-aired during this exemplary method, the inflow cannula can be connected directly to the heart through the sheath with no risk of air emboli remaining in the ventricle. As another option, the VAD can be connected at any point and the de-airing can be performed from the VAD outflow graft.

When the VAD is connected, the sheath (in embodiments including a sheath) is left in the ventriculotomy. This can provide additional sealing (e.g. for greater safety) between the ventriculotomy and the device inflow cannula. In certain embodiments, the sheath can be a foldable unit (e.g. having a breakable, peeling off, tearing off design). The surgical operation is then finalized according to standard technique.

Regarding specific details, according to an exemplary method of the present invention, the LV apex is approached and cannulated according to the Seldinger technique (i.e. a needle is inserted into the LV apex and then a guidewire is introduced into the LV which is followed with a larger cannula insertion). As another option, the guidewire can be directly inserted into the ventricle. As another option, direct cannulation (without a needle and/or a guidewire) can be performed. The occlusion system is introduced through the cannula into the LV. Once introduced into the LV, the distal occluder and subsequently the proximal occluder are dilated/inflated, thus providing a mechanical barrier to the blood coming to the LV apex area. This provides a bloodless surgical field and allows for direct visualization of the surgical field. When both occluders are dilated, there is no blood flow through these mechanical barriers and the surgeon is able to core the LV apex with a coring knife in the bloodless field. At the same time, the patient's physiological circulation through the heart is not altered and the patient's systemic hemodynamics is not compromised.

In current practice of LV coring without embodiments of an occlusion system, there is no visualization of the surgical field because of the large amount of blood coming out from the hole created in the LV. As such, under current practice, there is a chance to not resect enough trabeculae (cardiac tissue inside the ventricular chamber), which may obstruct the flow into the LVAD inflow cannula. Aspects of occlusion systems of the present invention can provide better visualization of the trabeculae through the hole in the LV. Thus, a surgeon can more accurately resect the trabeculae since the surgeon can see this tissue more clearly. Similarly, if VAD replacement is indicated, there is no technique to visualize the heart chamber after removing a previous device and inserting a new one.

As stated above, embodiments of systems, kits, and methods of the present invention provide direct visualization of the surgical field and intracardiac structures, such as intraventricular structures, during implantation of a VAD or other mechanical circulatory support device. Certain embodiments of systems provide for seamless VAD implantation, which reduces the risks and time associated with VAD implantation. Further, employing disclosed methods can avoid CPB. Another advantage of disclosed embodiments is that such embodiments provide a true bloodless field with no compromise to a patient's physiological circulation and systemic hemodynamics. Disclosed embodiments also do not necessarily require any additional incisions or peripheral vessel catheterizations. All manipulations can be performed through the main incision (such as a sternotomy or thoracotomy, or other procedure). If less invasive approaches are used, a transcatheter insertion and operation of the device can occur. Disclosed systems can be compatible with existing surgical techniques and do not necessarily require alteration of standard VAD implantation procedures.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A cardiac chamber visualization kit comprising:
an occlusion system comprising:
a catheter having a distal portion and a proximal portion and at least one lumen extending therein;
a distal occluder mounted on the distal portion of the catheter; and
a proximal occluder mounted on the proximal portion of the catheter; and
a mechanical circulatory support device.

2. The cardiac chamber visualization kit of claim 1, wherein the distal occluder is a distal expandable member and the proximal occluder is a proximal expandable member.

3. The cardiac chamber visualization kit of 2, wherein the distal expandable member is a distal balloon and the proximal expandable member is a proximal balloon.

4. The cardiac chamber visualization kit of claim 1, wherein the at least one lumen of the catheter comprises:
a first lumen in fluid communication with an infusion source at one end and the distal occluder at another end; and
a second lumen in fluid communication with the infusion source at one end and the proximal occluder at another end.

5. The cardiac chamber visualization kit of claim 1, wherein the lumen comprises:
a first infusion line extending in the lumen, the first infusion line in fluid communication with an infusion source at one end and the distal occluder at another end;
a second infusion line extending in the lumen, the second infusion line in fluid communication with the infusion source at one end and the proximal occluder at another end.

6. The cardiac chamber visualization kit of claim 2, wherein the distal expandable member, the proximal expandable member, or both has a substantially ring-shaped configuration surrounding a mesh or fabric material.

7. The cardiac chamber visualization kit of claim 2, wherein the distal expandable member, the proximal expandable member, or both is substantially cylindrical shaped.

8. The cardiac chamber visualization kit of claim 1, further comprising a sheath configured to fit in the hole of a cardiac wall of a patient.

9. The cardiac chamber visualization kit of claim 1, wherein the mechanical circulatory support device is a ventricular assist device.

10. A method of visualizing a cardiac chamber for insertion or removal of a mechanical circulatory support device in a patient comprising:
providing an occlusion system comprising:
a catheter having a distal portion and a proximal portion and at least one lumen extending therein;
a distal occluder mounted on the distal portion of the catheter; and
a proximal occluder mounted on the proximal portion of the catheter;
introducing a cannula into a cardiac chamber of the patient through a cardiac chamber wall;
inserting the occlusion system into the cannula with the distal and proximal occluders in a contracted state;
introducing the distal and proximal occluders into the cardiac chamber;
expanding the distal and proximal occluders sequentially or simultaneously into an operative configuration;
coring tissue of the cardiac chamber wall;
removing the cored tissue of the cardiac chamber wall;
de-airing the cardiac chamber;
removing the distal and proximal occluders from the cardiac chamber;
connecting a mechanical circulatory support device to the cored cardiac chamber wall via an inflow cannula.

11. The method of claim 10, wherein a sheath is placed in the cored cardiac chamber wall and the inflow cannula is connected to the sheath.

12. The method of claim 10, wherein the cardiac chamber is a ventricle.

13. The method of claim 12, wherein the cardiac chamber is a left ventricle.

14. The method of claim 10, wherein the cardiac chamber wall is a ventricular apex.

15. The method of claim 14, wherein the ventricular apex is the left ventricular apex.

16. The method of claim 10, wherein the mechanical circulatory support device is a ventricular assist device.
